(19) United States
(12) Patent Application Publication
(10) Pub. No.: US 2021/0315975 A1
(43) Pub. Date: Oct. 14, 2021

(54) METHOD FOR PREVENTION OR TREATMENT OF OBESITY, REDUCING BODY WEIGHT AND/OR FOOD INTAKE, OR INDUCING SATIETY IN A SUBJECT

(71) Applicant: Benjamin Lewes Woolven, Kanimbla (AU)

(72) Inventor: Benjamin Lewes Woolven, Kanimbla (AU)

(21) Appl. No.: 17/230,602

(22) Filed: Apr. 14, 2021

(30) Foreign Application Priority Data
Apr. 14, 2020 (AU) 2020901183

(51) Int. Cl. A61K 38/26; A61N 1/36; A61K 9/00

(52) U.S. Cl. CPC A61K 38/26; A61K 9/0019; A61N 1/36014

(57) ABSTRACT

A method for prevention or treatment of obesity, reducing body weight and/or food intake, or inducing satiety in a subject, the method comprising the steps of: administration of an effective amount of a GLP-1 agonist to the subject for an initial time period; positioning an electrode adjacent to and spaced from a skin surface of a head of the subject; and applying an electrical impulse through the electrode to a target region in a cerebral cortex of a brain of the patient, wherein the electrical impulse is sufficient to modulate one or more neurons in the target region.

### TECHNICAL FIELD

The present invention relates to a method for prevention or treatment of obesity, reducing body weight and/or food intake, or inducing satiety in a subject.

### BACKGROUND

Any references to methods, apparatus or documents of the prior art are not to be taken as constituting any evidence or admission that they formed, or form part of the common general knowledge.

Obesity is a complex and multifactorial condition characterized by adipose tissue accumulation that is strongly associated with multiple comorbidities, including type 2 diabetes, cancer, cardiovascular diseases, sleep apnea, and physical and social limitations. Calorie-restricted diets associated with lifestyle intervention are the first-choice treatment for weight loss. However, weight loss through hypocaloric diets could induce neuroendocrine changes that trigger an intense, subconscious, and powerful urge to eat, known as food craving. It is known that finding and consuming food can provide a rewarding feeling in obesity sufferers and this behaviour can result in failure to maintain a hypocaloric diet.

Although diet and exercise provide a simple process to decrease weight gain, overweight and obese individuals often cannot sufficiently control these factors to effectively lose weight. Pharmacotherapy is available; several weight loss drugs have been approved by the Food and Drug Administration that can be used as part of a comprehensive weight loss program. However, many of these drugs have serious adverse side effects. When less invasive methods have failed, and the patient is at high risk for obesity related morbidity or mortality, weight loss surgery is an option in carefully selected patients with clinically severe obesity. However, these treatments are high-risk, and suitable for use in only a limited number of patients. It is not only obese subjects who wish to lose weight. People with weight within the recommended range, for example, in the upper part of the recommended range, may wish to reduce their weight, to bring it closer to the ideal weight. Thus, a need remains for methods that can be used to effect weight loss in overweight and obese subjects without resorting to surgical intervention.

### SUMMARY OF INVENTION

In an aspect, the invention provides a method for prevention or treatment of obesity, reducing body weight and/or food intake, or inducing satiety in a subject, the method comprising the steps of:

administration of an effective amount of a GLP-1 agonist to the subject for an initial time period;

positioning an electrode adjacent to and spaced from a skin surface of a head of the subject; and

applying an electrical impulse through the electrode to a target region in a cerebral cortex of a brain of the patient, wherein the electrical impulse is sufficient to modulate one or more neurons in the target region.

In an embodiment, the GLP-1 agonist has a half-life of at least 1 hour.

In an embodiment, the GLP-1 agonist is administered in an amount of at least 0.7 mg per week.

In an embodiment, the GLP-1 agonist is administered at a molar equivalent amount of no more than 2.2 nmol per kg body weight of the subject.

In an embodiment, said GLP-1 agonist has a half-life of at least 24 hours, at least 96 hours, at least 120 hours, or at least 144 hours.

In an embodiment, the GLP-1 agonist has an EC₅₀ at or below 3000 pM, such as at or below 500 pM or at or below 100 pM.

In an embodiment, the GLP-1 agonist is administered in an amount of i) at least 0.8 mg, at least 1.0 mg, or at least 1.2 mg, such as at least 1.4 mg or at least 1.6 mg, per week.

In an embodiment, the wherein said GLP-1 agonist is selected from the group consisting of semaglutide, exenatide, albiglutide, and dulaglutide.

In one embodiment an “effective amount” of a GLP-1 agonist as used herein means an amount sufficient to cure, alleviate, or partially arrest the clinical manifestations of a given disease or state and its complications. An amount adequate to accomplish this is defined as “effective amount”. Effective amounts for each purpose will depend on the severity of the extent of obesity and the general state of the subject. It will be understood that determining an appropriate dosage may be achieved using routine experimentation, by constructing a matrix of values and testing different points in the matrix, which is all within the ordinary skills of a trained physician. In one embodiment the term “treatment” or “treating” as used herein means the management and care of a patient for the purpose of combating a condition, such as a disease or a disorder. In one embodiment the term “treatment” or “treating” is intended to include the full spectrum of treatments for a given condition from which the patient is suffering, such as administration of the active compound to alleviate the symptoms or complications; to delay the progression of the disease, disorder, or condition; to alleviate or relieve the symptoms and complications; and/or, to cure or eliminate the disease, disorder, or condition as well as to prevent the condition. In one embodiment prevention is to be understood as the management and care of a patient for the purpose of combating a disease, condition, or disorder and includes the administration of the active compounds to prevent the onset of the symptoms or complications.

In one embodiment the term “GLP-1 peptide” as used herein means GLP-1 (7-37), a GLP-1 analogue, a GLP-1 derivative or a derivative of a GLP-1 analogue.

In an embodiment, the stimulation step is carried out at a pre-determined stimulation treatment frequency over a pre-determined stimulation treatment period before commencement of the surgical procedure.

In an embodiment, the pre-determined stimulation treatment period ranges between two days and 28 days and more preferably between 5 days and 21 days and more preferably between 7 days and 14 days.

In an embodiment, the stimulation treatment frequency comprises providing at least one stimulation treatment session for at least 5 minutes and more preferably at least 10 minutes and still more preferably in the range of 15 minutes and 25 minutes wherein at least one of said treatment session is conducted on the patient over 3 or more consecutive days during the treatment period.

In an embodiment, at least 3 and preferably at least 5 treatment sessions are conducted on the patient over said consecutive days during the treatment period.

In an embodiment, the target region comprises the frontal lobes of the patent more specifically the right dorsolateral parieto-frontal cortex (R DLPFC).

In an embodiment, the stimulation treatment comprises a dosage of 1-3 and more preferably at least 2 milli amperes of current during every treatment session.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is a diagram describing the positioning of the anodal and cathodal electrodes over the right and left dorsolateral prefrontal cortex regions of the brain.

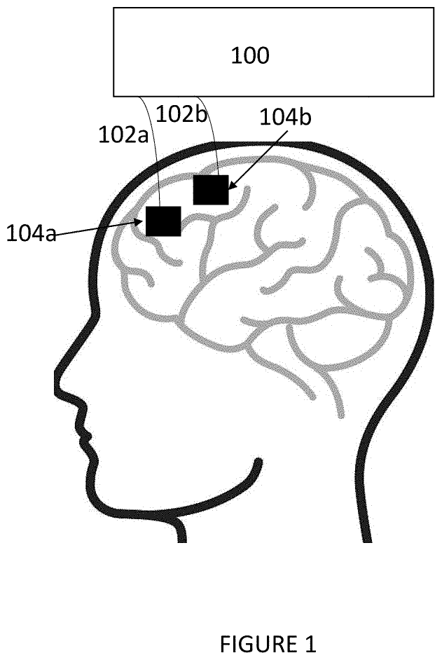

### DETAILED DESCRIPTION OF PREFERRED EMBODIMENTS

The presently described embodiments of the invention relate to an improved method for use of GLP-1 agonists in combination with non-invasive neuro-stimulation for suppression of food cravings by long terms neuromodulation.

In a first functional aspect, the GLP-1 agonists of the invention have a good potency. Also, or alternatively, in a second functional aspect, the GLP-1 agonists of the invention have a protracted pharmacokinetic profile. Also, or alternatively, in a third functional aspect, the GLP-1 agonists of the invention are stable against degradation by gastro intestinal enzymes. Biological activity (potency)

According to the first functional aspect, the GLP-1 agonists of the invention are biologically active, or potent. In a particular embodiment, “potency” and/or “activity” refers to in vitro potency, i.e. performance in a functional GLP-1 receptor assay, more in particular to the capability of stimulating cAMP formation in a cell line expressing the cloned human GLP-1 receptor.

In one embodiment the term half maximal effective concentration (EC50) generally refers to the concentration which induces a response halfway between the baseline and maximum, by reference to the dose response curve. EC50 is used as a measure of the potency of a compound and represents the concentration where 50% of its maximal effect is observed.

The in vitro potency of the GLP-1 agonists of the invention may be determined as described above, and the EC50 of the GLP-1 agonist in question determined. The lower the EC50, the better the potency.

In a particular embodiment, the medium may have the following composition (final in-assay concentrations): 50 mM TRIS-HCl; 5 mM HEPES; 10 mM MgCl2, 6H20; 150 mM NaCl; 0.01% Tween; 0.1% BSA; 0.5 mM IBMX; 1 mM ATP; 1 pM GTP; pH 7.4.

In a further particular embodiment, the GLP-1 agonist of the invention has an in vitro potency corresponding to an EC5o at or below 3000 pM, such as below 2000 pM, below 1000 pM, or below 500 pM, or such as below 200 pM or below 100 pM.

In another particular embodiment the GLP-1 agonist of the invention are potent in vivo, which may be determined as is known in the art in any suitable animal model, as well as in clinical trials.

In some embodiments, GLP-1 agonists of the invention which include non-natural amino acids and/or a covalently attached N-terminal mono- or dipeptide mimetic may e.g. be produced as described in the experimental part. Or see e.g., Hodgson et al: “The synthesis of peptides and proteins containing non-natural amino acids”, Chemical Society Reviews, vol. 33, no. 7 (2004), p. 422-430; and WO 2009/083549 A1 entitled “Semi-recombinant preparation of GLP-1 analogues” and these references have been incorporated entirely into the description.

In at least one exemplary embodiment, the method for treating or preventing obesity, for reducing body weight and/or food intake, or for inducing satiety comprises administration of a GLP-1 agonist to a subject in need thereof in an amount of at least 0.7 mg per week, such an amount equivalent to at least 0.7 mg semaglutide per week. The GLP-1 agonist has a half-life of at least 24 hours, such as at least 48 hours, at least 60 hours, or at least 72 hours, or such as at least 84 hours, at least 96 hours, or at least 108 hours, or optionally at least 120 hours, at least 132 hours, or at least 144 hours, wherein said half-life optionally is determined by Assay (II). The GLP-1 agonist may be administered twice weekly or less often, once weekly or less often, such as less often than once weekly or once every secondly week or less often, or such as once every third week or less often or once a month or less often. The GLP-1 agonist may be administered in an amount of at least 0.8 mg, at least 10.0 mg, or at least 10.2 mg, such as at least 10.4 mg or at least 10.6 mg, per week. The GLP-1 agonist may be administered in an amount equivalent to at least 0.8 mg, at least 10.0 mg, or at least 1.2 mg, such as at least 1.4 mg or at least 1.6 mg, semaglutide per week.8. In some embodiments, the said GLP-1 agonist is administered by parenteral administration, such as subcutaneous injection. The GLP-1 agonist is administered simultaneously or sequentially with another therapeutic agent.

In one embodiment the GLP-1 peptide comprises the amino acid sequence of the formula (I)

Xaa₇-Xaa₈-Glu-Gly-Thr-Phe-Thr-Ser-Asp-Xaa₁₆-Ser-Xaa₁₈-Xaa₁₉Xaa₂₀GluXaa₂₂-Xaa₂₃-Ala-Xaa₂₅-Xaa₂₆-Xaa₂₇-Phe-Ile-Xaa₃₀-Trp-Leu-Xaa₃₃-Xaa₃₄-Xaa₃₅-Xaa₃₆-Xaa₃₇-Xaa₃₈-Xaa₃₉-Xaa₄₀-Xaa₄₁-Xaa₄₂-Xaa₄₃-Xaa₄₄-Xaa₄₅-Xaa₄₆ Formula (I)

wherein
Xaa₇ is L-histidine, D-histidine, desamino-histidine, 2-amino-histidine, β-hydroxy-histidine, homohistidine, N^{α}-acetyl-histidine, α-fluoromethyl-histidine, α-methyl-histidine, 3-pyridylalanine, 2-pyridylalanine or 4-pyridylalanine;
Xaa₈ is Ala, Gly, Val, Leu, Ile, Lys, Aib, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid;
Xaa₁₆ is Val or Leu;
Xaa₁₈ is Ser, Lys or Arg;
Xaa₁₉ is Tyr or Gln;
Xaa₂₀ is Leu or Met;
Xaa₂₂ is Gly, Glu or Aib;
Xaa₂₃ is Gln, Glu, Lys or Arg;
Xaa₂₅ is Ala or Val;
Xaa₂₆ is Lys, Glu or Arg;
Xaa₂₇ is Glu or Leu;
Xaa₃₀ is Ala, Glu or Arg;
Xaa₃₃ is Val or Lys;
Xaa₃₄ is Lys, Glu, Asn or Arg;
Xaa₃₅ is Gly or Aib;
Xaa₃₆ is Arg, Gly or Lys;
Xaa₃₇ is Gly, Ala, Glu, Pro, Lys, amide or is absent;
Xaa₃₈ is Lys, Ser, amide or is absent;
Xaa₃₉ is Ser, Lys, amide or is absent;
Xaa₄₀ is Gly, amide or is absent;
Xaa₄₁ is Ala, amide or is absent;
Xaa₄₂ is Pro, amide or is absent;
Xaa₄₃ is Pro, amide or is absent;
Xaa₄₄ is Pro, amide or is absent;
Xaa₄₅ is Ser, amide or is absent;
Xaa₄₆ is amide or is absent;
provided that if Xaa₃₈, Xaa₃₉, Xaa₄₀, Xaa₄₁, Xaa₄₂, Xaa₄₃, Xaa₄₄, Xaa₄₅ or Xaa₄₆ is absent then each amino acid residue downstream is also absent.

In another embodiment the GLP-1 peptide comprises the amino acid sequence of formula (II)

Xaa₇-Xaa₈-Glu-Gly-Thr-Phe-Thr-Ser-Asp-Val-Ser-Xaa₁₈-Tyr-Leu-Glu-Xaa22-Xaa₂₃-Ala-Ala-Xaa₂₆-Glu-Phe-Ile-Xaa₃₀-Trp-Leu-Val-Xaa₃₄-Xaa₃₅-Xaa₃₆-Xaa₃₇Xaa₃₈ Formula (II)

wherein
Xaa₇ is L-histidine, D-histidine, desamino-histidine, 2-amino-histidine, -hydroxy-histidine, homohistidine, N^{α}-acetyl-histidine, α-fluoromethyl-histidine, α-methyl-histidine, 3-pyridylalanine, 2-pyridylalanine or 4-pyridylalanine;
Xaa₈ is Ala, Gly, Val, Leu, Ile, Lys, Aib, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid;
Xaa₁₈ is Ser, Lys or Arg;
Xaa₂₂ is Gly, Glu or Aib;
Xaa₂₃ is Gln, Glu, Lys or Arg;
Xaa₂₆ is Lys, Glu or Arg; Xaa30 is Ala, Glu or Arg;
Xaa₃₄ is Lys, Glu or Arg;
Xaa₃₅ is Gly or Aib;
Xaa₃₆ is Arg or Lys;
Xaa₃₇ is Gly, Ala, Glu or Lys;
Xaa₃₈ is Lys, amide or is absent.

GLP-1 agonist may be administered before or after commencing a neurostimulation treatment such as transcranial direct current stimulation (TDCS). Preferably, the GLP-1 agonist may be administered to the subject over an initial administration period such as at least 3 days, more preferably at least one week and still more preferably at least two weeks before commencing a neurostimulation treatment such as TDCS. TDCS is a non-invasive neuromodulation technique that provides a safe, painless, inexpensive method to induce neuroplasticity. In the presently described method the applicant combines neuromodulation with administering an effecting amount of a GLP-1 agonist to improve weightloss outcomes in the subject. As will be evident from the following sections, it is hypothesized that dorsolateral prefrontal cortex (DLPFC) neuromodulation of a patient by way of TDCS after commencing the step of administering an effective amount of the above-described GLP-1 agonist for an initial time period may control the desire to eat and thus may be involved in food intake regulation which consequently improves outcomes and possibly prevent the need for surgical intervention such as bariatric surgery.

In general, TDCS uses a relatively constant, low flow of direct electrical current delivered to the scalp, directly over the target brain area of a patient, using small electrodes. More particularly, when the electrodes are placed in the regions of interest specifically the right Dorsolateral prefrontal Cortex (R DLFPC), electrical current flow through the electrodes induces intracerebral current flow. This intracerebral current flow can either increase or decrease neuronal excitability in specific areas being stimulated based on which type of stimulation is being used. This change of neuronal excitability leads to alteration of brain function, and it is understood by the applicant that the neuron excitability of the right DLFPC after commencement of treatment with the GLP-1 agonist can lead to reduced food cravings.

In the preferred embodiment, the TDCS stimulation step involves the use of an exemplary TDCS device 100 connected via a pair of electrical cables 102a, 102b with electrodes 104 a, 104 b at their respective distal ends to the right DLFPC region in the head of a human patient 106. In the illustrated example, a first one of the electrodes 104 a is positioned near a first frontal portion of the patient's head in the DLFPC region and a second one of the electrodes 104 b is positioned back on the top of the patient's head, displaced at least several centimetres away from the first electrode 104 a. Of course, effective TDCS may be achieved with a variety of different electrode configurations.

In a typical implementation, before the TDCS is initiated, a human operator would program into the TDCS device 100 at least: 1) a target value of current, and 2) a duration for delivering the target value of current to the human patient 106. Initial work done by the applicant suggests that a preferred target value of 2 milli amperes for a duration of approximately 20 minutes in each treatment session is likely to be beneficial. The applicant has also found that preferably 3 to 5 treatment sessions a week preferably over a two-week period is likely to provide beneficial effects by reducing food cravings when such treatment is carried out over a treatment period of 1 to 2 weeks. It is important to note that after the subject has commenced GLP-1 agonist treatment for at least one week and preferably two weeks, the TDCS neuro-stimulation should be carried in the above described manner whilst the subject is still consuming the GLP-1 agonist medication in this neuro-stimulation treatment period of 1 to 2 weeks. It has been hypothesized that the combination of the TDCS treatment after initial commencement of GLP-1 agonist medication maximise the effect of the treatment in achieving long term neuromodulation while the brain is in a ketotic state induced by the decreased caloric intake achieved by the GLP-1 agonist. In addition, after an initial TDCS treatment step, the subject may continue to be administered with the effective amount of the GLP-1 agonist over a prolonged period such as 12-16 weeks, the neuro-stimulation treatment as described above may be repeated. Again, it is hypothesized that the subsequent TDCS treatment maximise the probability of achieving long term neuromodulation via lasting changes in synaptic function while the brain is in a hunger suppressed state as a result of decreased caloric intake and gut hormonal changes induced by the GLP-1 agonist.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

1. A method for prevention or treatment of obesity, reducing body weight and/or food intake, or inducing satiety in a subject, the method comprising the steps of:
administering an effective amount of a GLP-1 agonist to the subject for an initial time period;
positioning an electrode adjacent to and spaced from a skin surface of a head of the subject; and
applying an electrical impulse through the electrode to a target region in a cerebral cortex of a brain of the patient, wherein the electrical impulse is sufficient to modulate one or more neurons in the target region.

2. A method in accordance with claim 1 wherein the GLP-1 agonist has a half life of at least 1 hour.

3. A method in accordance with claim 1 wherein the GLP-1 agonist is administered in an amount of at least 0.7 mg per week.

4. A method in accordance with claim 1 wherein the GLP-1 agonist is administered at a molar equivalent amount of no more than 2.2 nmol per kg body weight of the subject.

5. A method in accordance with claim 1 wherein said GLP-1 agonist has a half-life of at least 24 hours, at least 72 hours, at least 96 hours, at least 120 hours, or at least 144 hours.

6. A method in accordance with claim 1 wherein the GLP-1 agonist has an EC₅₀ at or below 3000 pM.

7. A method in accordance with claim 1 wherein the GLP-1 agonist is administered in an amount of at least 0.8 mg per week.

8. A method in accordance with claim 1 wherein the wherein said GLP-1 agonist is selected from the group consisting of semaglutide, exenatide, albiglutide, and dulaglutide.

9. A method in accordance with claim 1 wherein the initial stimulation step is carried out at a pre-determined stimulation treatment frequency over a pre-determined stimulation treatment period before commencement of the surgical procedure.

10. A method in accordance with claim 9 wherein the pre-determined stimulation treatment period ranges between two days and 28 days and more preferably between 5 days and 21 days and more preferably between 7 days and 14 days.

11. A method in accordance with claim 9 wherein the stimulation treatment frequency comprises providing at least one stimulation treatment session for at least 5 minutes, wherein at least one of said treatment session is conducted on the patient over 3 or more consecutive days during the treatment period.

12. A method in accordance with claim 11 wherein at least 3 treatment sessions are conducted on the patient over said consecutive days during the treatment period.

13. A method in accordance with claim 12 wherein the target region comprises the frontal lobes of the patent more specifically the right dorsolateral parieto-frontal cortex (R DLPFC).

14. A method in accordance with claim 1 wherein the stimulation treatment comprises a dosage of 1-3 milli amperes of current during every treatment session.

15. The method in accordance with claim 1 wherein the GLP-1 agonist has an EC₅₀ at or below 500 pM.

16. The method in accordance with claim 1 wherein the GLP-1 agonist has an EC₅₀ at or below 100 pM.

17. The method in accordance with claim 9 wherein the pre-determined stimulation treatment period ranges between 5 days and 21 days.

18. The method in accordance with claim 9 wherein the pre-determined stimulation treatment period ranges between 7 days and 14 days.

19. The method in accordance with claim 9 wherein the stimulation treatment frequency comprises providing at least one stimulation treatment session in the range of 15-25 minutes.

20. The method in accordance with claim 11 wherein at least 5 treatment sessions are conducted on the patient over said consecutive days during the treatment period.
